(19) United States
(12) Patent Application Publication
(10) Pub. No.: US 2019/0040484 A1
(43) Pub. Date: Feb. 7, 2019

(54) METHOD FOR FACILITATING THERMOMECHANICAL FORMING PROCESS OF AUSTENITE CONTAINING GRADES TO PRODUCE TAILORED STRENGTH STRUCTURAL COMPONENTS

(71) Applicant: AK Steel Properties, Inc., West Chester, OH (US)

(72) Inventor: Kavesary S Raghavan, West Chester, OH (US)

(21) Appl. No.: 16/051,929

(22) Filed: Aug. 1, 2018

Related U.S. Application Data
(60) Provisional application No. 62/539,921, filed on Aug. 1, 2017.

(51) Int. Cl. C21D 8/10; C21D 8/00

(52) U.S. Cl. CPC C21D 8/105; G01N 33/20; C21D 8/005

(57) ABSTRACT

A method to quantitatively determine the amount of deformation induced martensite as a function of temperature and strain in austenitic stainless steels is used to customize the strength and elongation characteristics of certain portions of a formed structural component. Predicting the martensitic volume fraction in a specific part location permits design of particular components with customized strength characteristics that can be consistently repeatably manufactured.

### PRIORITY

This application claims priority to U.S. Provisional Application Ser. No. 62/539,921, entitled “A Method for Facilitating Thermomechanical Hydro/Sheet Forming Process of Austenite containing Grades to Produce Tailored Strength Structural Components,” filed on Aug. 1, 2017, the disclosure of which is incorporated by reference herein.

### BACKGROUND

The automotive industry continually seeks more cost-effective steels that are lighter for more fuel-efficient vehicles and stronger for enhanced crash-resistance, while still being formable. Tailored strength structural components are essential to achieve weight-saving while meeting structural performance of automotive components. Today, there is increasing use of tailored press hardened components in the marketplace that manipulate the ability to control thermal martensite via controlled cooling following hot stamping.

Applications of tailored strength components can include various rail sections (front rail, roof rails, rear rails) that can potentially be produced through thermo-mechanical tube hydroforming process as well as B-pillars and sheet formed components that are produced using segmented cooled/heated dies to achieve specific strength levels in different locations.

Austenitic steels typically have higher ultimate tensile strengths combined with high total elongations. The austenitic microstructure is ductile and has the potential to produce high total tensile elongations. The austenitic microstructure is sometimes not stable at room temperatures (or is metastable), and when the steel is subjected to plastic deformation the austenite often transforms into martensite (stress/strain induced martensite). Martensite is a microstructure with higher strengths, and the combined effect of having a mixture of microstructures, such as austenite plus martensite, is to increase of the overall tensile strength. When austenite is subjected to plastic deformation and transforms to martensite, the overall strength of the steel is increased.

Next generation steels (and metastable austenitics) rely on austenite transformation to martensite with deformation for strengthening. Steel grades suitable for a quench and partitioning process, or those that exhibit transformation induced plasticity, can be carefully processed to achieve, for example, 10-20% retained austenite. The expectation is that this austenite will transform to martensite during forming to provide additional strength in the finished part. Austenite stability is known to have dependence on steel composition, temperature, strain rate, strain level, and stress state. Increasing the temperature suppresses transformation and increases austenite stability. Increasing the strain rate results in higher adiabatic heat generation in the sample and effectively has the same effect as increasing the test temperature. In general, increasing the strain level promotes transformation with a sigmoidal dependence. In any complex stamped part, the deformation mode can range from draw conditions (pure shear to plane strain) to stretching conditions (plane strain to balanced biaxial tension). Martensitic transformation occurs through pure shear, and there is literature which suggests that even at the same equivalent strain the amount of transformation may vary with the deformation mode.

### BRIEF DESCRIPTION OF THE FIGURES

FIG. 1 depicts an isothermal stress/strain curve for Nitronic 30 austentic steel produced and sold by AK Steel Corporation, West Chester, Ohio.

FIG. 2 depicts the martensite volume fraction versus strain for the Nitronic 30 material.

FIG. 3 depicts martensite volume fraction as a function of temperature for the Nitronic 30 material.

FIG. 4 depicts the characteristic strain curve fit to be an exponential curve as a function of temperature for the Nitronic 30 material.

FIG. 5 depicts experimental data of volumetric fraction of martensite as function of temperature for the Nitronic 30 material.

FIG. 6 depicts the martensite volume fraction vs. normalized strain for the Nitronic 30 material.

FIG. 7 depicts experimentally measured transformation data for several austenitic stainless steels.

FIG. 8 depicts the best fit curve of the data set forth in FIG. 7.

FIG. 9 depicts a generic B-pillar from an automobile.

FIG. 10 depicts a generic front crush rail for an automobile.

### DETAILED DESCRIPTION

The present embodiments facilitate the development of thermo-mechanical hydroforming or sheetforming processes for producing structural components from austenite-containing stainless and carbon grades with tailored properties by controlling the amount of deformation martensite in a given part location by controlling the temperature and strain introduced during forming.

The present application pertains to a methodology that can be used to facilitate rapid design of thermo-mechanical processes (tube hydroforming, sheet hydroforming, and/or conventional sheet metal forming) in order to produce tailored strength structural components. In many of these components, it may be advantageous to tailor the strength in different regions of the component.

The methods described in this present application can be used to quantitatively predict the amount of deformation induced martensite as a function of temperature and strain that has been proven to work very well for austenitic stainless steels and use that calculation to customize the strength and elongation characteristics of certain portions of the structural component. Predicting the martensitic volume fraction in a specific part location will permit design of particular components with customized strength characteristics that can be consistently repeatably manufactured. The method described here can easily be adapted to other austenite bearing carbon/stainless steel grades that rely on transformation induced plasticity (TRIP) mechanism.

It is well known that metastable austenitic stainless steels and next generation retained austenite-containing advanced high strength steels rely on transformation of austenite during deformation for strengthening. As explained above, the transformation kinetics are believed to depend upon the chemical composition, strain amount, temperature (strain rate) and possibly deformation mode.

Empirical measures of austenite stability such as M_{d}30 and Instability Factor are available that provide directional guidance on how stable or unstable a given austenitic grade is based on chemical composition. M_{d}30 is defined as the temperature at which 50% martensite is present at 30% strain. M_{d}30 can be calculated using the following equation:

M_{d}30(° C.)=413-462*(C+N)−9.2*Si−8.1*Mn−13.7*Cr−9.5*Ni−18.5*Mo Eqtn. 1

Typically, the higher the M_{d}30 value, the more unstable the austenite in the grade.

An instability function (IF) was introduced in a U.S. Pat. No. 3,599,320 for assessing austenite stability within a specific range of chemical composition containing 0.07-0.18 C, 0.9-6.2 Mn, 4.1-7.7 Ni, 14.1-17.9 Cr, 0.01-0.14 N with the balance being iron. The instability function varying from 0 to 2.9 was reported for this composition range, and determined by the equation (2) below:

IF=37.193−51.248[C]−1.0174[Mn]−2.5884[Ni]−0.4677[Cr]−34.396 [N] (Eqtn. 2)

Steels that exhibited IF values between 0 and 2.9 were classified as being “slightly metastable” in the patent filing, while “wholly stable” steels exhibited negative IF values.

However, the methods to define austenite stability are not useful for quantitative prediction of the amount of martensite based on a given strain and temperature at a particular location on a formed component. M_{d}30 and IF will tell you whether a grade with a particular composition is more or less stable. That is, a material with lower austenite stability will transform more (higher volume fraction of martensite) compared to a material that has high austenite stability.

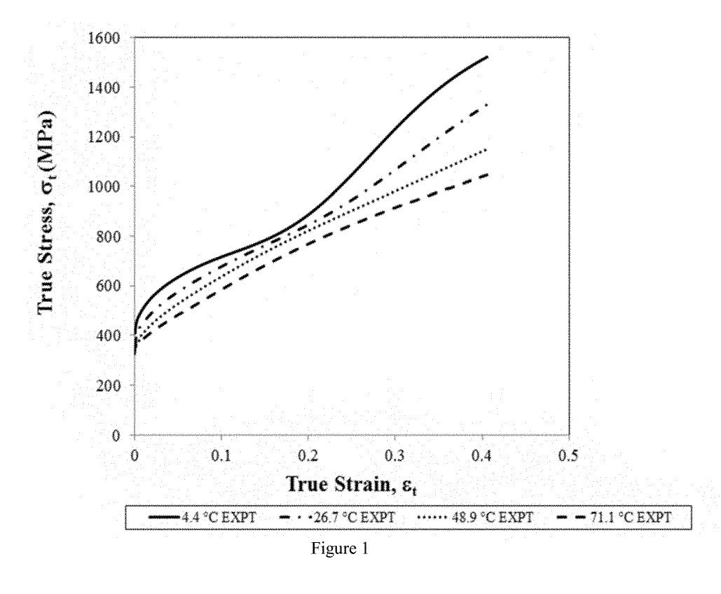

The present methods provide a more coherent methodology that can be used not only to indicate austenite stability of a particular grade, but also to design thermo-mechanical processes (hydroforming or conventional forming) to produce tailored strength levels in different structural components. As an example of the potential enhancement in strength level possible with temperature control, data from isothermal tests is shown for a Nitronic 30 steel grade in FIG. 1; at a true strain of 0.4, true stress increases to 1500 MPa at a temperature of 4.4° C. while at 71.1° C., true stress is only 1000 MPa.

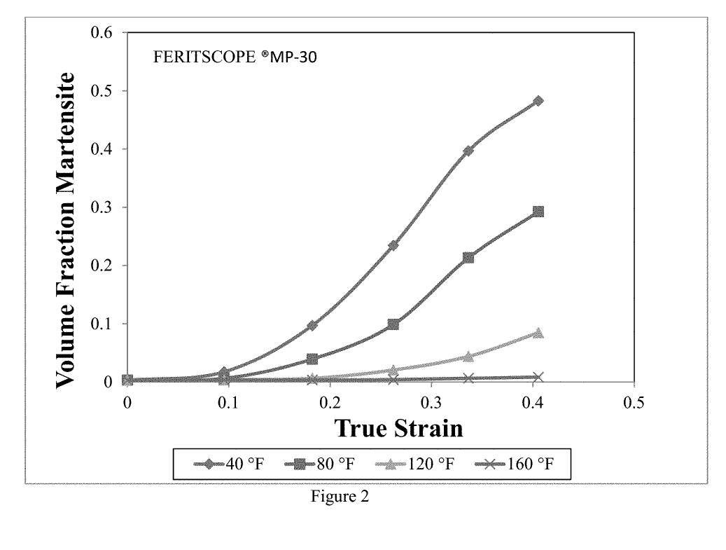

The inflection in stress strain response is a result of deformation induced martensite that can be measured with interrupted testing to different strain levels using several techniques including magnetic induction, X-ray diffraction or Neutron diffraction. Data from these different methods are related to each other via linear extrapolation methods; an example of measured amount of deformation martensite using magnetic induction is shown in FIG. 2 for the same Nitronic 30 steel data described in FIG. 1.

While the data in FIG. 2 can be experimentally generated at any temperature of interest and can be used to pick out the volume fraction of deformation induced martensite, this can become a very cumbersome process requiring several experiments for each austenitic stainless grade of interest at all temperatures of interest.

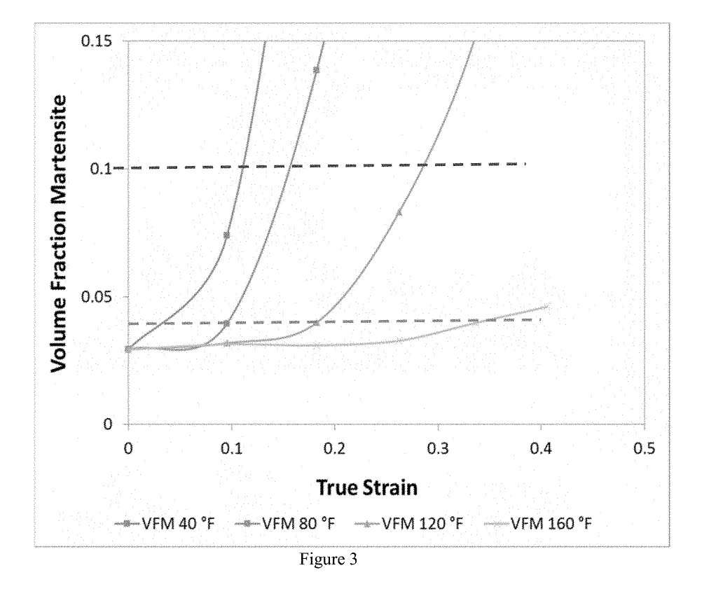

To streamline this understanding of austenite transformation kinetics into a useful predictive tool, a characteristic strain curve methodology was developed to normalize the data with respect to temperature and strain level. It is to be noted that the effect of strain rate is implicitly included in this approach since an increasing strain rate corresponds to more adiabatic internal heating in the deformed sample that results in an increase in temperature. In the present methods, the characteristic strain is simply the amount of strain needed to achieve a desired (arbitrary) amount of transformed martensite at different temperatures. An example is shown in FIG. 3 for the same Nitronic 30 steel material corresponding to data in FIGS. 1 and 2 for a choice of either 0.10 volume fraction of martensite or closer to 0.04 volume fraction of martensite. The choice of volume fraction of martensite is arbitrary and simply a matter of convenience depending on the austenite stability of the particular grade.

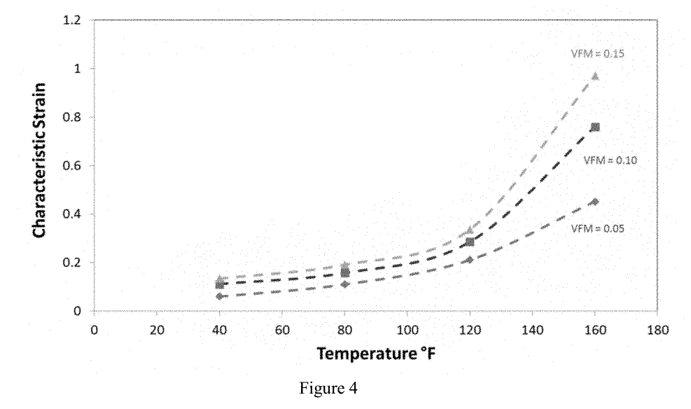

Characteristic strain curves for the Nitronic 30 material based on different choices of amount of martensite is shown in FIG. 4. The characteristic strain curve can be fit to an exponential curve as a function of temperature; this allows us to extrapolate information from tensile tests conducted at a few temperatures to a wide range of temperatures of interest in thermo-mechanical forming (hydro/sheet) processes.

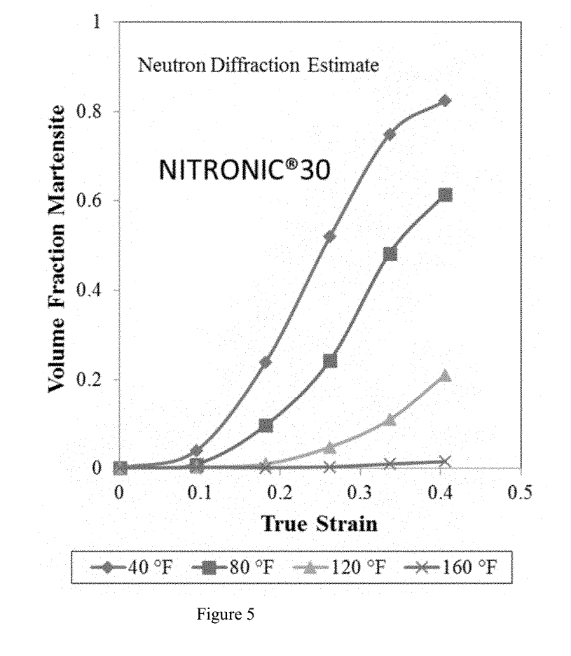

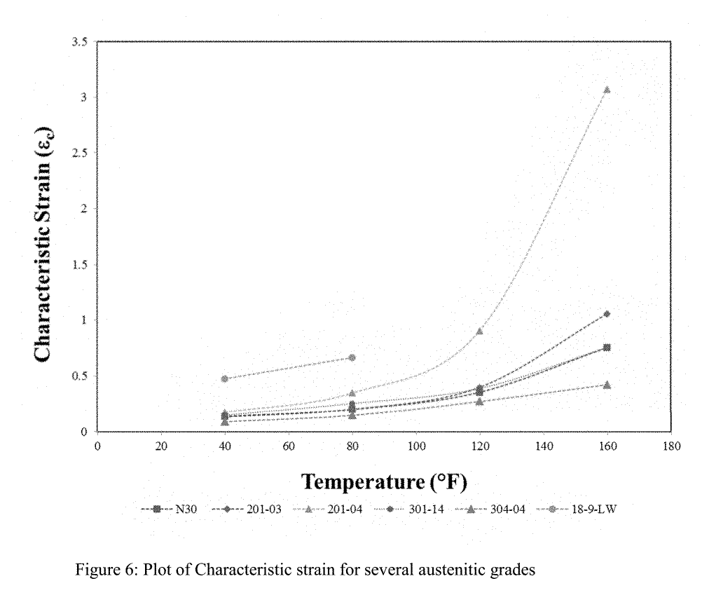

Once the characteristic strain curve is known for a given grade, it allows us to predict the volume fraction of martensite in any thermo-mechanical process for that particular austenite containing grade as shown in FIGS. 5 and 6 for the Nitronic 30.

The single curve shown in FIG. 6 not only encompasses the experimental data in FIG. 5 but can be used to predict the volume fraction of martensite at any desired temperature and strain level for this Nitronic 30 grade.

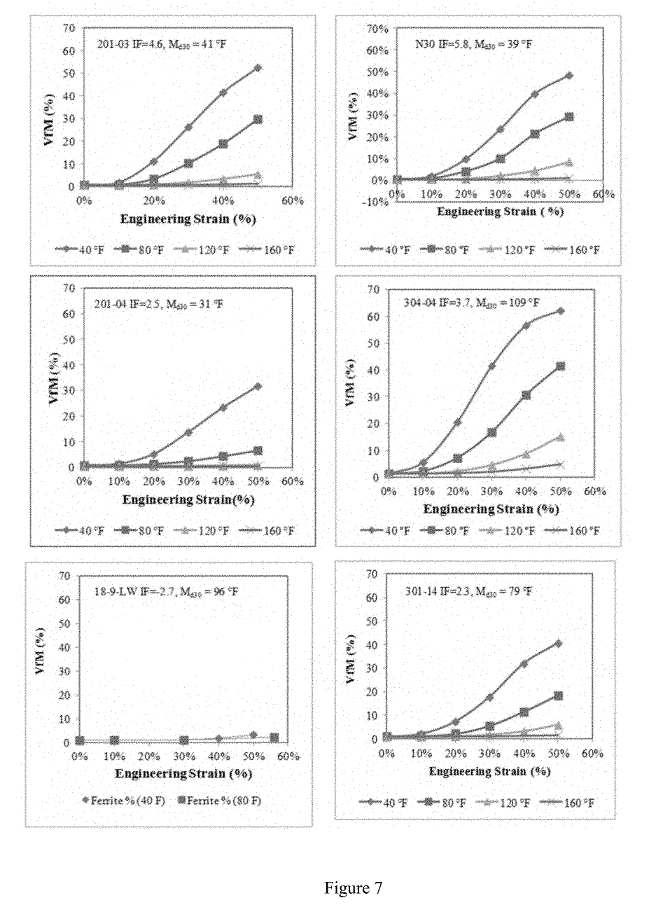

A further simplication occurs in the case of austenitic stainless steels. In this case, we have discovered that a single universal curve exists that is sufficient to describe the amount of transformed martensite in all these grades. FIG. 7 below shows the experimentally measured transformation data for a wide variety of austenitic stanless steels.

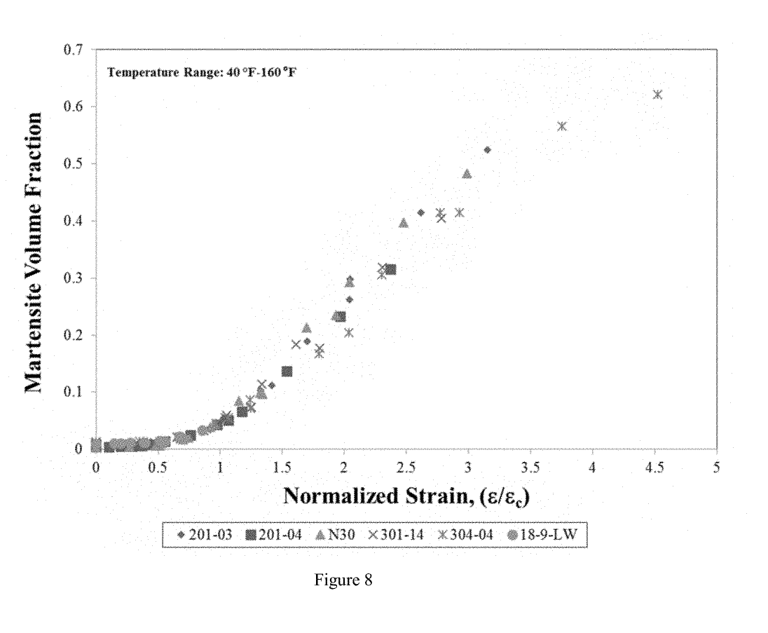

We have discovered that all of the austenitic stainless steel data maps into one single universal transformation curve shown in FIG. 8; with the knowledge of the characteristic strain curve that is unique to each austenitic grade, we can simply determine the volume fraction of martensite at any desired temperature and strain level in a thermo-mechanical process. This information is important for rapidly designing components with tailored strength levels in different regions in any thermo-mechanical process.

The present process provides the following benefits. This universal curve can be used for austenitic steels, or any steels that contain retained austentite, to quantitatively predict volume fraction martensite in a given part for a given grade, or to obtain a desired volume fraction martensite in a given part.

Although the equations were developed in uniaxial tension, they can also be developed to be used in the same manner for different modes, such as biaxial tension. And the above described process can be used to develop similar curves for other steel materials.

### Example 1: Conventional Sheet Forming with Segmented Dies

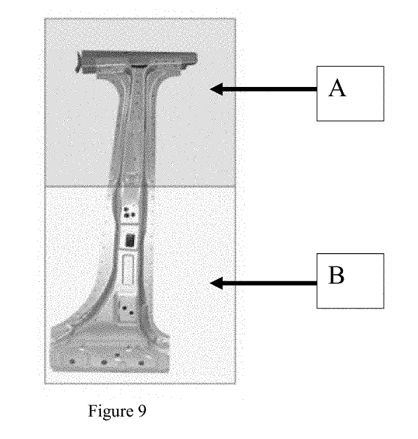

An exemplary part, a generic representation of an automotive B-pillar, depicted in FIG. 9, is produced commonly in a conventional stamping operation involving placement of a blank within a die set that includes a blankholder, a punch and a mating die. The blankholder is moved first to constrain the movement of the blank into the die and the punch is then moved to make the part. An alternative approach to make the same component is to use a sheet hydroforming process, where instead of using a punch, fluid pressure is used to drive the blank into the die.

The method described herein facilitates the thermo-mechanical forming in conventional sheet forming with segmented dies, or with sheet hydroforming with temperature control for a generic B-pillar such as the one in FIG. 9. The method comprises the following process:
1. Designer needs higher strength (for example 1200 MPa) to resist intrusion in the top of part (part A in FIG. 9) but needs a lower strength (for example 800 MPa) higher ductility bottom region (part B in FIG. 9) for energy absorption.
2. To reach those goals, for instance in the case of NITRONIC 30, FIG. 1 shows that 1200 MPa can be achieved at a true strain of 0.3 at a temperature ˜0 C (the line reflects results at 4.4 C). Similarly, you get an 800 MPa strength level at a strain of 0.2 at a temperature of close to 0 C.
3. Now, of course if the designer could try and set up the stamping process to use 0 C and then design the die to vary the “effective” strain (note, that in a sheet metal part there are in-plane principal strains but these can be converted to a single equivalent effective strain) to be 0.3 at the top of the B-pillar and 0.2 in the bottom section, then he will achieve the desired strength differential. In general, this type of brute force approach will be infeasible because the specific temperature of 0 C or the ability to vary strain from 0.3 on the top to 0.2 on the bottom cannot be achieved.
4. In embodiments of the present process, we recognize first that strength in austenitic steel is directly related to volume fraction of martensite and austenite. So for this austenitic steel NITRONIC 30 steel, we find from FIG. 2 that 0.2% strain at 0 C corresponds to ˜10% volume fraction of martensite measured using a magnetic FERITSCOPE, and 0.3% strain corresponds to ˜30% volume fraction of martensite. (Please note if phase volume fractions are measured with XRD, the martensite volume fraction would be 1.73×volume fraction measured with magnetic FERITSCOPE method.)
5. Here is where the elegance of our methodology comes in. Using the universal curve of FIG. 8, for a desired level of transformation we can pick off normalized strain (ε/ε_{c}) at 30% martensite and 10% martensite. The curve will show that ε/ε_{c} ˜2.4 for 30% martensite and ε/ε_{c} ˜1.2 for 10% martensite. (Please note again that the volume fraction martensite reported here are based on magnetic FERITSCOPE method and these should be scaled by a factor of 1.73 to correlate with XRD values.)
6. The (ε/ε_{c}) values of 1.2 for 10% martensite volume fraction and 2.4 for 30% martensite is the same value independent of grade and temperature for all austenitic steels.
7. The process designer now knows the target (ε/ε_{c}) for a specific target volume fraction of martensite in different regions of the component to achieve the tailored strength levels he is seeking.
8. Depending on part geometry, he has some control over the strain distribution he can achieve in different regions. Finite element analysis can give him predictive capability for the strains he can achieve in different regions of the part.
9. Once the strains (in both principal directions and converted to an effective strain) achievable in a given geometry is known, and the target ε/ε_{c} is known, the only variable left is the characteristic strain value.
10. The characteristic strain value is a function of the grade and the temperature. FIG. 6 shows characteristic strain plots for several common austenitic grades.
11. The process designer can have two options at this point. If he chooses to have a monolithic component made out of one material, he can use FIG. 6 to find the temperature differential he needs, he can develop the appropriate thermo-mechanical forming (tube/sheet hydroforming or conventional sheet forming with segmented dies) to create the tailored strength component for the application.

The second option the process designer has is to say that he does not want to alter the temperature as much and will choose to use a welded blank with two different materials that transform differently to achieve the targeted strength differential he is seeking to create a tailored strength component. The concept invoked here is that a given volume fraction of martensite (corresponds to a given target strength level) is directly related to the normalized strain WO as shown in FIG. 8. The normalized strain has two components—the effective strain on the part and the characteristic strain which depends on the grade's austenite stability and the temperature. Please note that austenite stability is just the propensity of transformation potential in the material. At a given strain and temperature, material with low austenite stability would transform more and result in higher volume fraction of martensite in the particular region.

### Example 2: Tube Hydroforming of Front Crush Rail Component

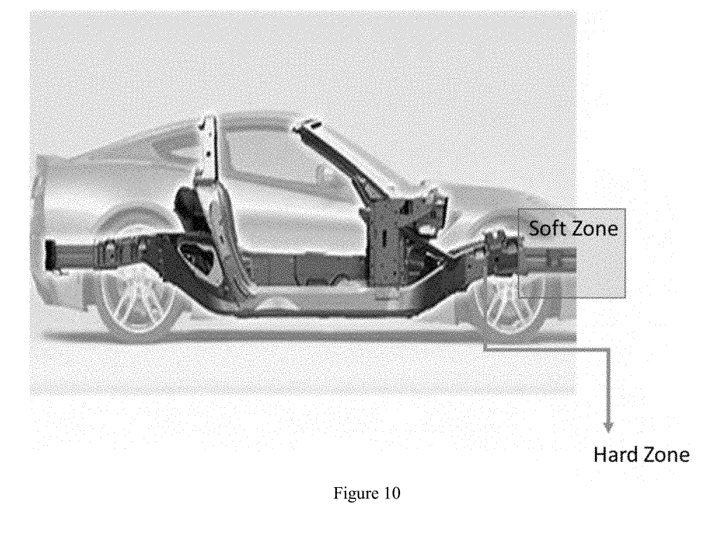

A second exemplary part is a front crush rail component such as shown in FIG. 10. Part design intent could be to have lower strength in the front end to allow it to absorb energy during frontal impact but at some point in the crush process there should be no further deformation to prevent collapse into the passenger compartment. So the rear end of the part should have high strength.
i. Front soft zone and a harder rear zone
ii. The consideration and approach is the same up to step 9 in the Example 1 for conventional sheet forming with segmented dies.
iii. An issue with tube (or sheet) hydroforming is that it can be difficult to change the temperature in different zones in the hydroforming process with one fluid.
iv. Option 1: Use a monolithic tube. Perform hydroforming in two stages—the front of the part with one temperature of the fluid and then do the rear part with a different temperature of the fluid, each selected based on the date of FIG. 8.
v. Option 2: Construct a tailor welded tube with two different austenite grades—the front part from a material with high austenite stability and the rear part with material with low austenite stability.
vi. Option 3: Use a monolithic tube—change to a larger section in the rear part so that more strain will be achieved during hydroforming and therefore more transformation in the rear section at the same temperature.
vii. Any of the options defined above will result in a tailored strength component.

### Example 3

A formed part is made by identifying at least one pre-determined mechanical property in a region of the formed part; associating a martensite volume fraction level with said pre-determined strength level; using a universal strain curve to determine the normalized strain corresponding to the martensite volume fraction; and either selecting a steel to provide the normalized strain or selecting process constraints in a forming process to provide the normalized strain.

### Example 4

The process of Example 3, or any following example, wherein the normalized strain is provided by selecting a particular grade of steel.

### Example 5

The process of Examples 3 or 4, or any following example, wherein the normalized strain is provided by selecting process constraints in a forming process.

### Example 6

The process of Examples 3, 4, or 5, or any following example, wherein the process constraints comprise at least one of effective strain or forming temperature.

### Example 7

The process of Examples 3, 4, 5, or 6, or any following example, wherein the effective strain is provided by a stamping die configuration.

### Example 8

A blank made of steel is formed into a formed part, wherein the formed part has at least two regions with differing mechanical properties; by identifying the mechanical parameters for each region of the part; associating a martensite volume fraction with the identified mechanical parameters for each region of the part; determining a characteristic strain curve for the steel; based on the characteristic strain curve for the steel, selecting the true strain and temperature necessary to create the martensite volume fraction associated with each region of the part; configuring a die to provide the selected true strain for each region of the part; and forming each region of the part at the forming temperature and in the die configuration selected for said region to create the tailored formed part.

### Example 9

The process of example 8, or any following examples, wherein the steel is an austenitic stainless or carbon steel.

### Example 10

The process of example 8 or 9, of any following examples, wherein the steel includes retained austenite.

### Example 11

The process of examples 8, 9, or 10, or any following examples, wherein the blank is a tube.

### Example 12

The process of examples 8, 9, 10, or 11, or any following examples, wherein the blank is a sheet.

### Example 13

The process of examples 8, 9, 10, 11, or 12, or any following examples, wherein the mechanical properties can include strength level.

### Example 14

The process of examples 8, 9, 10, 11, 12, or 13, or any following examples, wherein the characteristic strain curve for the steel is determined by identifying the amount of austenite transformed into martensite after deformation at three or more different temperatures.

### Example 15

A formed part is formed by providing a blank made of steel to be formed into a formed part, wherein the formed part has at least two regions with differing mechanical properties; identifying the mechanical parameters for each region of the part; associating a martensite volume fraction with the identified mechanical parameters for each region of the part; determining a characteristic strain curve for the steel; based on the characteristic strain curve for the steel, selecting the true strain and temperature necessary to create the martensite volume fraction associated with each region of the part; configuring a die to provide the selected true strain for each region of the part; and forming each region of the part at the forming temperature and in the die configuration selected for said region to create the tailored formed part.

### Example 16

The process of example 16 wherein the characteristic strain curve for the steel is determined by identifying the amount of austenite transformed into martensite after deformation at three or more different temperatures.

What is claimed is:

1. A process for preparing a formed part comprising the steps of:
a. Identifying at least one pre-determined mechanical property in a region of the formed part;
b. Associating a martensite volume fraction level with said pre-determined mechanical property;
c. Using a universal strain curve to determine the normalized strain corresponding to the martensite volume fraction; and
d. Either selecting a steel to provide the normalized strain or selecting process constraints in a forming process to provide the normalized strain.

2. The process of claim 1 wherein the normalized strain is provided by selecting a particular grade of steel.

3. The process of claim 2 wherein the normalized strain is provided by selecting process constraints in a forming process.

4. The process of claim 3 wherein the process constraints comprise at least one of effective strain or forming temperature.

5. The process of claim 4 wherein the effective strain is provided by a stamping die configuration.

6. The process of claim 1 wherein the steel is an austenitic stainless or carbon steel.

7. The process of claim 1 wherein the steel includes retained austenite.

8. The process of claim 1 wherein the blank is a tube.

9. The process of claim 1 wherein the blank is a sheet.

10. The process of claim 1 wherein the mechanical properties can include strength level.

11. A process for preparing a formed part comprising the steps of:
a. Providing a blank made of steel to be formed into a formed part, wherein the formed part has at least two regions with differing mechanical properties;
b. Identifying the mechanical parameters for each region of the part;
c. Associating a martensite volume fraction with the identified mechanical parameters for each region of the part;
d. Determining a characteristic strain curve for the steel;
e. Based on the characteristic strain curve for the steel, selecting the true strain and temperature necessary to create the martensite volume fraction associated with each region of the part;
f. Configuring a die to provide the selected true strain for each region of the part;
g. Forming each region of the part at the forming temperature and in the die configuration selected for said region to create the tailored formed part.

12. The process of claim 11 wherein the characteristic strain curve for the steel is determined by identifying the amount of austenite transformed into martensite after deformation at three or more different temperatures.
